(19) United States
(12) Patent Application Publication
(10) Pub. No.: US 2018/0228107 A1
(43) Pub. Date: Aug. 16, 2018

(54) IN VITRO CULTURE METHOD TO INCREASE THE BIOMASS AND INCREASE THE NUMBER OF VARIETY PLANTS AND PRUNUS SPP INJERT HOLDER, PARTICULARLY PRUNUS AVIUM

(71) Applicants: INSTITUTO DE INVESTIGACIONES AGROPECUARIAS, La Florida, RM (CL); BIOTECNOLOGÍA FRUTÍCOLA S.A., Vitacura, RM (CL)

(72) Inventors: Eduardo Andrés Tapia Rodríguez, La Florida, RM (CL); Sebastián Ignacio Godoy González, Macul, RM (CL); Humberto Godofredo Prieto Encalada, Vitacura, RM (CL)

(73) Assignee: BIOTECNOLOGÍA FRUTÍCOLA S.A., Vitacura, RM (CL)

(21) Appl. No.: 15/816,251

(22) Filed: Nov. 17, 2017

(30) Foreign Application Priority Data
Nov. 18, 2016 (CL) 02945-2016

(51) Int. Cl. A01H 4/00; A01H 6/74; A01H 5/10; C12N 5/04

(52) U.S. Cl. CPC A01H 4/005; C12N 5/04; A01H 5/10; A01H 6/7427

(57) ABSTRACT

The present invention is an in vitro culture method to increase both the biomass and the number of seedlings in a number varieties and rootstock types of Prunus spp., particularly Prunus avium, and the use of said culture method, which is a useful means of micropropagating in vitro any variety and rootstock type of Prunus spp., in particular Prunus avium.

The current invention belongs to an in vitro culture method to increase the biomass and increase the number of seedlings of varieties and rootstocks for Prunus spp., particularly Prunus avium.

The in vitro culture method involves the following stages:
a) Collecting shoots from the donor plant,
b) Washing the shoots with water for 10-30 minutes and disinfecting them with a 50% commercial bleach solution for 20-40 minutes,
c) Submerging the shoots in a pesticide for 20-40 minutes,
d) Submerging the shoots in an indolebutyric acid solution (IBA) for 20-40 minutes,
e) Collecting the buds and introducing them into a temporary immersion system made up of the media A and B, wherein immersions are performed between 2 and 8 times per day with a duration of 1 to 6 minutes for the duration of the 10-20 days culture period.
f) The in vitro seedlings are obtained after the required culture period.

### STATE OF THE ART BACKGROUND

There are many techniques and methods for the culture and micropropagation of plant tissues. Regardless of that, all of them take into account certain common stages: selection of the original donor plant, collection and disinfection of explants, adaptation of the explants to the culture medium, root formation to obtain seedlings, acclimatization of the seedlings to real environmental conditions. Among these stages, one of the key and deciding issues of success for the micropropagation is the adaptation of the explants to the culture medium; as such, several factors need to be considered such as medium type, exposure time to the medium, temperature, light, amongst other factors.

To solve the issue of the shoots exposition to the culture medium for longer times, but in a controlled manner, there is the existence of the so called temporary immersion bioreactors or temporary immersion system. These are automatic or semiautomatic systems used as tools for the in vitro propagation and mass increase of plant tissues. Overall, this type of system is made up of two culture flasks connected amongst themselves by a hose connection type, where one of the flasks corresponds to the physical space for explants growth and the other one contains the culture medium. The hose connected between the two containers allows the movement of the culture medium.

The propagation systems based on an immersion system have several advantages regarding the propagation of shoots in solid media and in liquid media with constant exposition (not included in temporary immersion systems): they avoid the continuous immersion of the shoots, this protects the plant material from growth and morphogenesis issues due to the constant exposition to a medium that in addition to containing the nutrients would also contain the metabolic waste generated; they facilitate the flow and transference of oxygen; they provide a more efficient supply, and they allow to alternate between several custom culture media; the risk of contamination due to the constant manipulation is reduced, and is less expensive, as it requires lower amounts of media since they are pulse-fed (Teisson C, Alvard D, Lartaud M, Etienne H, Berthouly M, Escalona M. y Lorenzo J C (1999) Temporary immersion for plant tissue culture. In: Plant Biotechnology and In vitro Biology in the 21^{st} Century, Proceedings of the IXth International Congress of Plant Tissue and Cell Culture, Section H: Novel micropropagation methods (pp 629-632)). Even though it is possible to micropropagate cultures by an immersion system, their complexity lies in the precise election of the propagation culture medium, their exposure times, and the number of pulses, since each species and rootstock have unpredictable growth conditions that are complex to determine.

In the agricultural industry, the cherry crop (Prunus avium) represents a significant part of the export fruit production. As such, its handling and reproduction should consider the suitable conditions to produce a fruit that maintains its conditions during the exportation time.

The growth of cherry trees depends, just like most plant species, in the variety, the use or lack of use of rootstocks, and the environmental conditions (water, light, nutrients, and soil type). However, in comparison to other species, the cherry branches in a lesser degree since its ramification has particular features which hinder or slow down the process. The cherry ramification is generated from the formation of verticils, which corresponds to the ramifications exclusively formed in the distal section from the shoots from the previous year, the lack of ramifications over growths from the same year and furthermore, the cherry ramification is produced by its ability to reshoot from latent subjects.

In general, the cherry culture and propagation conditions have been kept constant over time without the existence of many new varieties. Traditionally, the growth of Prunus varieties in nurseries is performed with the following procedure: multiplication by axillary ramification, elongation, root penetration and acclimatization of the seedling. The problems related to this common work practice in nurseries is that each stage needs to be adjusted to the particular type of Prunus genotype where the hyperhydricity, apex necrosis, unstable propagation, and root penetration parameters need to be controlled.

Additionally, the micropropagation of the Prunus species in in vitro has also been described. For example, the scientific dissemination document from Nemazy E. et al. presents protocols to optimize the species micropropagation from the Prunus specimens from GF677 micro-shoots. For their growth, the micro-shoots were cultivated in a MS medium supplemented with 0.5% pectin, 0.75 mg/L 6-benzylaminopurine (hereinafter BAP), 0.01 mg/L indolebutyric acid (hereinafter IBA), 20 g/L sucrose, and 7 g/L agar (Nemaziy E. et al., Plant Cell Tiss Organ Cult (2014) 117:349-359). This document mainly refers to the Prunus micropropagation in a solid medium, without making any reference to a continuous system based on liquid media with specific features and components.

Studies like the one from Sulusoglu M. have the objective to improve the micropropagation procedure in vitro of Prunus laurocerasus (cherry laurel), a native species from the western area of the Black Sea, Turkey, where its commercial exploitation is not relevant yet. As such, we propose a consistent protocol to sterilize shoots and cultivate them in a Murashige y Skoog (MS) medium which contains different concentrations of 6-benzylaminopurine (BAP) and in some cases the addition of indole-3-butyric acid (IBA). From that we obtained a higher number of shoots when treated with a medium of 20. mg/L of BAP combined with 0.1 mg/L of IBA. The process involves routines of several successive transplants for four weeks with renewal of the nutritional medium (Melekber Sulusoglu y Aysun Cavusoglu, Food, Agriculture and Environment (JFAE) Vol. 11, Issue 1, pages 576-579).

Other documents also reference the Prunus spp micropropagation in a culture medium where the main components are IBA and BAP. As such, the document with patent CN101822217 presents a preparation method of a culture medium for big Gisela type cherry. The method comprises two culture media, one a differentiation medium based on benzylaminopurine and one for root penetration containing indole-3-butyric. On the other hand, document CN101161058 presents a propagation method of shoots from cherry rootstock, indicating that the culture medium derives from the method containing IBA.

The scientific paper from Dulic, J. et al. presents the assessment of a culture of embryos in-ovule and ex-ovule condition of Prunus avium when treated with a Murashige and Skoog culture medium supplemented with 1 mg/L of BA, 0.5 mg/L NAA, 20 g/L sucrose, 10 g/L sorbitol and 6 g/L agar. This culture medium improves the seeds' germination time, but belongs to a culture system in a solid medium. (Dulic, J., Ognjanov, V; Ercisli, S; Miodragovic, M; Barac, G; Ljubojevic, M.; Doric, D. (2016). In vitro Germination of Early Ripening Sweet Cherry Varieties (Prunus avium L.) at Different Fruit Ripening Stages. Erwerbs-Obstbau (2016) 58:113-118).

The document KR20150026554 (A) presents an efficient propagation methodology, root generation, adaptation and growth of Prunus avium clones. The growth protocol presented in this document includes the use of IBA in concentrations that range between 0.1 and 1.0 mg/L. In the case of the patent document CN 105379621 A, it presents a regeneration method for in vitro Prunus ‘Missy’ cherry plants, such method involves initially the induction of buds in a solid induction medium made of sucrose, agar, NAA and 1.0 mg/L of 6-benzyladenine, followed by subsequent root penetration stages.

Even though the aforementioned documents present the effect that treatment culture media that involve benzyladenine and indole-3-butyric over the Prunus avium shoots and/or embryos, these documents only reference the treatment with individual culture media, without any indication that these media are included in an immersion system with time variables and occasionally with controlled immersion to obtain the maximum micropropagation performance.

Other scientific papers present the assessment of the plant hormones benzyladenine (BA) and indole-3-butyric acid (IBA) on other Prunus species. For example, the scientific paper with the title “In vitro Propagation of ‘Garnem’ (P. persica×P. dulcis) Rootstock” from the work group of Sevde Kose and Fatih A. Canli, presents the evaluation of different types of cytokinin, such as benzyladenine (BA) and indole-3-butyric acid (IBA) over the in vitro multiplication for the ‘Garnem’ rootstock (P. persica×P. dulcis) (Sevde Kose and Fatih A. Canli. (2015). In vitro Propagation of ‘Garnem’ (P. persica×P. dulcis) Rootstock. J Plant Mol Biol Biotechnol 5(1): 25-30). Another paper from this investigation team, presents the research regarding the effect of benzyladenine and indole-3-butyric acid on the “Pixy” (Prunus institia L.) in vitro rootstock multiplication (Duygu Geyik and Fatih A. Canli (2015). Micropropagation of ‘Pixy’ (Prunus institia L.) Rootstock. J Plant Mol Biol Biotechnol 2015 5(1): 1-6). In both papers, the results show that the treatment with IBA and/or BA increases the number of roots and potentially the proliferation of rootstock. However, it is important to consider that even though the proposed media increase the number of roots and encourage proliferation, this type of methodology includes acclimatization stages and culture periods that at the end are longer than 30 days, and as such they are inefficient and time-consuming protocols.

Document CN101584299 discloses a culture medium for the propagation of Prunus serrulata Lindl, wherein such medium contains within its components BAP. Similarly, CN102090328 discloses a medium to enhance the cherry culture including BAP and IBA; however, this medium also contains agar, and as such is a propagation system in a solid medium. Thus, this technique has disadvantages like the depletion of the nutrients in the agar and the saturation of the waste products from the plant metabolism, as such it is a limited, less efficient protocol.

On the other hand, immersion systems in a liquid medium incorporate in their composition substances like BAP an IBA; however, they do not consider culture conditions such as immersion times and number of immersions that allow the maximum performance regarding biomass and seedlings production, and, overall, they are systems with very low performance regarding biomass production and number of plants. In this sense, the fact of proposing specific conditions and concentrations of the components to obtain a greater performance of Prunus micropropagation (number of immersions, immersion times, and minimum optimum concentrations for the culture media, reflect the technical effort carried out by the inventors. The selection of the optimal conditions for each variable and the combination of such in the same system is derived from the planning to adjust the variables and from performing repeated quantitative tests.

On the other hand, the systems and methods described up to this date conform to very slow culture methods that require more than 30 days to obtain a biomass and number of plants production performance comparable to the method proposed here. In this invention, the proposed methodology allows to obtain seedlings after 10-20 days from the introduction of explants in the temporary immersion system.

The proposed method includes a liquid immersion system with controlled variable, established to obtain a greater performance regarding the production of biomass and a greater number of Prunus spp seedlings.

### DESCRIPTION OF FIGURES

FIG. 1. Schematic figure of the immersion system. Immersion system scheme for the in vitro culture of different varieties of Prunus spp buds in 150 mL flasks containing 40 mL of propagation medium.

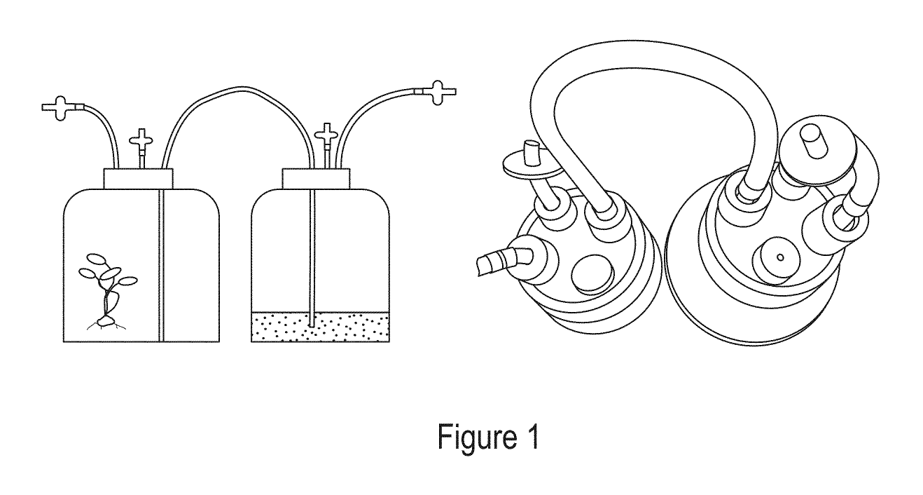

FIG. 2. Pictures of the initial material of segmented explants and effect on their growth when included in a temporal immersion system. Panel A shows the initial material for each Prunus spp variety; panel B shows the segmented explants from the initial material to be included in the temporary immersion system; panel C shows the explants after their exposure for 14 days in the temporary immersion system.

FIG. 3. Chart for multilevel parameters analysis immersion number (N), immersion time (T), and culture medium (M). Panel A shows the multilevel analysis for the different varieties considering the biomass production (Qx) as a parameter in relation to the number of immersions. Panel B shows the multilevel analysis for the different varieties considering the number of plants produced (Px) as a parameter in relation to the number of immersions.

FIG. 4. Chart for biomass production for the variety based on the immersion parameters applied. The chart shows the biomass production results for each variety (Maxma 14, Colt, Van and Rainier) in relation to the different immersion parameters, where T is the immersion time, N the number of immersions and m the type of culture medium, which could be culture medium A and culture medium B.

FIG. 5. Chart for number of seedlings produced for the variety based on the immersion parameters applied. The chart shows the seedlings production number results for each variety (Maxma 14, Colt, Van and Rainier) as per the different immersion parameters, where T is the immersion time, N the number of immersion and m the type of culture medium, which could be culture medium A and culture medium B.

FIG. 6. Chart of sucrose consumption for the variety based on the immersion parameters applied. The chart shows the sucrose consumption level results for each variety (Maxma 14, Colt, Van and Rainier) as per the different immersion parameters, where T is the immersion time, N the number of immersion and m the type of culture, medium which could be culture medium A and culture medium B.

### DESCRIPTION OF THE INVENTION

The current invention refers to an in vitro culture method to increase both the biomass and the number of seedlings of different Prunus spp varieties and rootstocks, particularly Prunus avium.

The in vitro culture method involves the following stages:
a) Collecting shoots from the donor plant,
b) Washing the shoots with water for 10 minutes and disinfecting them with a 50% commercial bleach solution for 20-40 minutes,
c) Submerging the shoots in a pesticide for 20-40 minutes,
d) Submerging the shoots in an indolebutyric acid solution (IBA) for 20-40 minutes,
e) Collecting the buds and introducing them into a temporary immersion system made up of media A and B, wherein immersions are performed 2 and 8 times per day with a duration of 1 to 6 minutes for the duration of the 10-20 days culture period.
f) The in vitro seedlings are obtained after the required culture period.

In this invention, whenever reference is made to the varieties and species of the Prunus genus, they correspond, but are not limited to: Prunus accumulans, Prunus africana, Prunus amplifolia, Prunus amigdaloides, Prunus amygdalus, Prunus annularis, Prunus argentea, Prunus besseyi, Prunus brachybotrya, Prunus brachypetala, Prunus brasiliensis, Prunus brittoniana, Prunus buxifolia, Prunus capollin, Prunus cerasifera, Prunus cercocarpifolia, Prunus chamissoana, Prunus cornifolia, Prunus debilis, Prunus detrita, Prunus divaricata, Prunus douglasii, Prunus emarginata, Prunus erythroxylon, Prunus espinozana, Prunus fasciculata, Prunus×ferganica, Prunus ferruginea, Prunus fortunensis, Prunus gentryi, Prunus glandulosa, Prunus guanaiensis, Prunus hainanensis, Prunus havardii, Prunus herthae, Prunus hintonii, Prunus huantensis, Prunus integrifolia, Prunus lanata, Prunus laurocerasus, Prunus leiocarpa, Prunus lichoana, Prunus ligus trina, Prunus lundelliana, Prunus mexicana, Prunus microphylla, Prunus moritziana, Prunus myrtifolia, Prunus nachichevanica, Prunus oblonga, Prunus oleifolia, Prunus omissa, Prunus opaca, Prunus ovalis, Prunus padifolia, Prunus pleiantha, Prunus prunifolia, Prunus pumila, Prunus punctata, Prunus ravenii, Prunus rhamnoides, Prunus rotunda, Prunus rufa, Prunus ruiziana, Prunus samydoides, Prunus sellowii, Prunus serotina, Prunus simonii, Prunus skutchii, Prunus spinosa, Prunus stipulata, Prunus subcoriacea, Prunus subcorymbosa, Prunus tetradenia, Prunus trichopetala, Prunus tucumanensis, Prunus ulei, Prunus urotaenia, Prunus ussuriensis, Prunus valida, Prunus virens, Prunus wurdackii.

In particular, it refers to Prunus avium, Prunus armeniaca, Prunus persica, Prunus mahaleb, Prunus insititia, Prunus dulcis, Prunus cerasus Prunus salicina and Prunus domstica.

The scope of this invention considers furthermore the varieties of Prunus, which could be, but are not limited to: Early burlat, Brooks, 210, Moreau, Tulare, Lapins, Rainer, 719, Garnet, Sweet Georgia, Black republican, Van, SHG, Sommerset, Vanda, Kordia, Stella, Hartland, Bing 260, Bing wab 13, Ruby, Van compact, Sunset bing, Black Tartarian, Sylvia, Sweet heart, NY 7690, Techlova, Karina, Summit, Regina, Celeste, Compact Stella, Napoleon, New star, Zirat 900, Sam, Hedelfingen, Sunburst, Lambert, Schmidt or Schneider.

Particularly, in stage c) of this method, the pesticide to use can be any pesticide capable of eliminating bacterial and fungal endogenous pathogens present in the explants; the preferred pesticide is benomile.

In stage 3) of the proposed method, the buds are collected and introduced in a temporary immersion system made up of the medium A and B, more details are included below:

| Medium A | Medium B |
|---|---|
| DKW base medium modified with | DKW base medium modified with |
| 0.1-0.001 mg/L of indole | 0.10 mg/L of indole butyric acid |
| butyric acid (IBA), 0.1-3 mg/L | (IBA), 0.6 mg/L of 6- |
| of 6-benzylaminopurine (BAP), | benzylaminopurine (BAP), 25 gr/L |
| 25 gr/L of sucrose, 0.5 gr/L of | of sucrose, 0.5 gr/L of |
| ascorbic acid and 0.1 gr/L of | ascorbic acid and 0.1 gr/L of |
| inositol | inositol |

The immersion system includes the culture media A and B, which—on the basis of a temporal immersion protocol consisting in 2 to 8 immersions per day lasting 1 to 6 minutes during the 10 to 20 culture days period—empower the growth of different Prunus genus varieties and rootstocks. The system activates in an automatic and controlled manner the output of the culture medium for the number of times and durations already indicated. We considered a total volume between 50 mL and 2 L for each culture medium.

After the bud incubation of 10 to 20 [days] in the temporal immersion system, it is possible to obtain in vitro seedlings.

To determine the seedlings' growth, it is suggested to evaluate the sucrose consumption, biomass production, and number of generated seedlings.

### Examples of the Application

### Example 1: In Vitro Culture Method to Increase the Biomass and Increase the Number of Seedlings from Prunus avium Genotypes in Comparison with the Micropropagation Protocol in Solid Medium

In this example, the best parameters and conditions to use in the temporal immersion bioreactor to obtain the greater growth or biomass increase in the following Prunus genotypes are presented: MAXMA-14 and Colt rootstock, and the Van and Rainier varieties. Concurrently with this, we compared the performance from different immersion protocols in relation to a micro propagation protocol from the varieties in a solid culture medium.

First, tissue portions from the genotypes submitted were collected, which were then washed with water for 10 minutes. Subsequently, the shoots were disinfected with 50% commercial bleach for 20 minutes, they were immersed in a 2% benomil solution for 20 minutes, and then stored at a temperature of 4° C. for 900 hours. Before planting the plant tissue, it was submerged in a solution with 1000 ppm of indolebutyric acid (IBA) for 20 minutes, and then planted in chambers at a temperature of 24° C. From their growth, the buds were collected and introduced in the in vitro culture using 150 mL flasks containing 40 mL of propagation medium. The temporal immersion system consisted of independent explants organized in a system of 10 bioreactors in a parallel setting.

The experiment was performed using a set of 100 seedlings of each genotype as the supply of explants: For this we evaluated the following immersion parameters:
Immersion time (T): 1 and 3 minutes.
Number of immersions (N): protocols of 2 and 4 immersions.
Culture medium (m): Culture medium A (0.01 mg/L IBA, 0.1 mg/L de BAP) and B (0.01 mg/L IBA, 0.6 mg/L BAP).

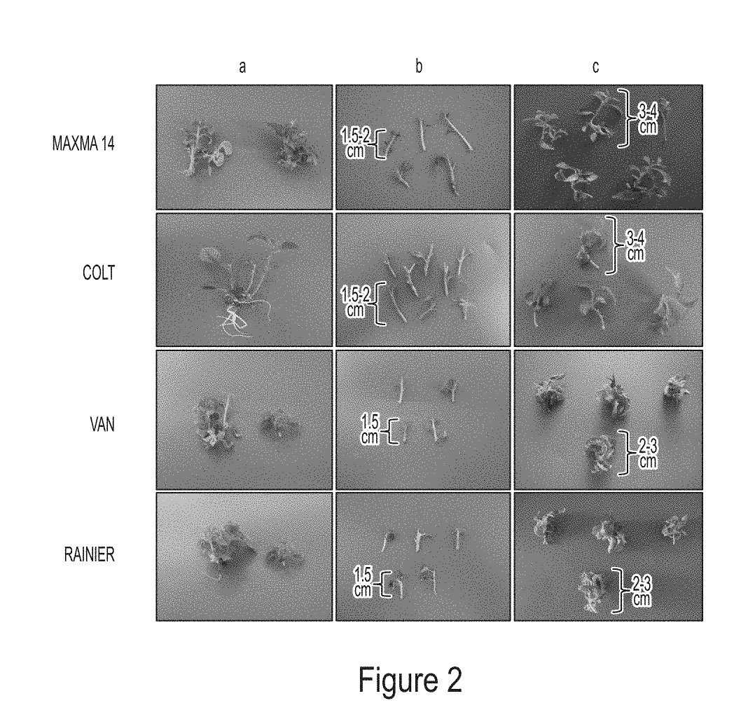

The explants used correspond to segmented explants from the initial material (FIG. 2, panels a and b). The explants in the temporary immersion system were withdrawn after 14 days of exposure, and it was observed that they had achieved a significant growth (FIG. 2, panel c).

To determine the performance of the explants exposure from the different varieties, we determined the parameters for sucrose consumption, the final biomass by weighing the material produced, and the number of resulting plants based on a recount. For the specific case of the sucrose consumption of the explants, we used the “Kit Sucrose Assay” (Sigma-Aldrich) following the manufacturer's instructions.

For the analysis of results, we applied multilevel factorial analysis, by means of which the best responses regarding the number of plants (PX) and the biomass (Qx) were determined.

Results of the Performance of the Immersion Parameters Evaluated

When the biomass (Qx) production was studied, the most important variable turned out to be the number of immersions.

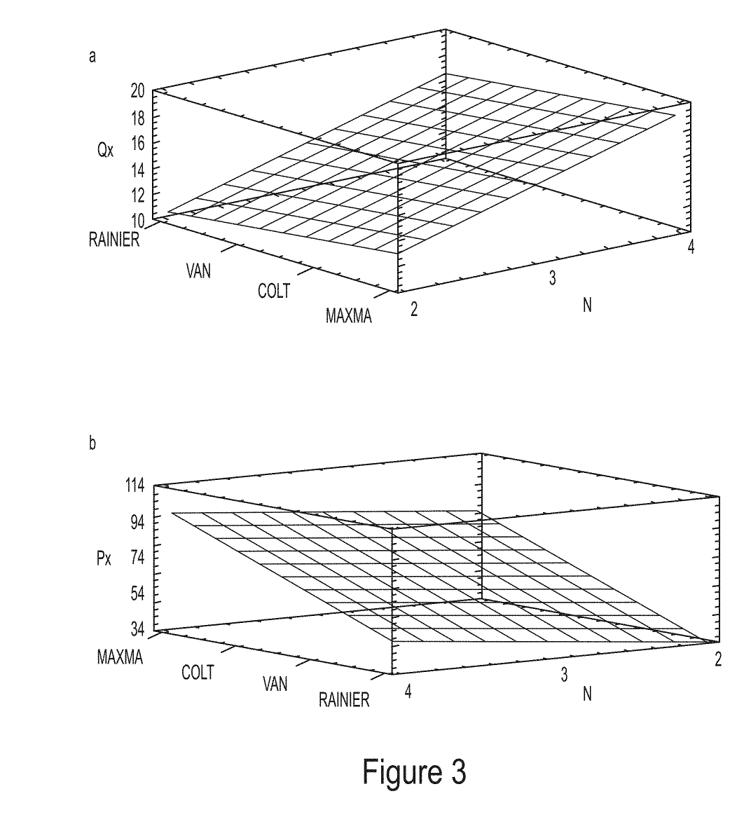

When applying a total of 4 times of immersion for 1 minute and using the medium B as propagation medium, the Maxma-14 genotype was the one with the greater biomass production with 23.85±1 g (FIG. 3a). Regarding the number of seedlings produced, we observed that the number of seedlings produced depended on the medium, the number of immersions, and the genotype used. Through a protocol comprising 3 or 4 immersions, for 1 or 3 minutes, and with medium B, we obtained the best results in explants from the MAXMA-14 genotype (117±10 explants) (FIG. 3b).

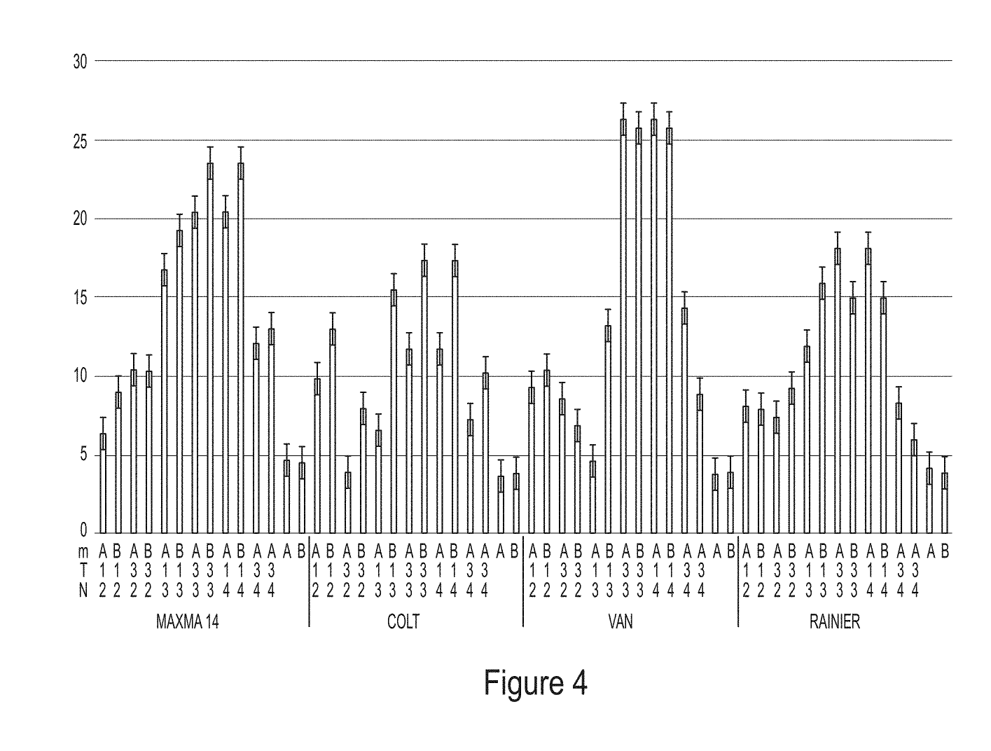

In the case of micropropagation of the MAXMA-14 variety, the immersion system made up by the medium B, with N=3 or 4 and T=1 or 3 min (FIG. 4; bars B,3,3 and B,1,4 in axis X) produced the highest biomass (23.85±1 g). For Colt we obtained the highest values using the medium A at N=4 and T=1 min (bar A.4.1) and medium B at N=3 and T=3 min (bar B.3.3); in both cases the production reached 17.3±1 g.

In the case of the ‘Van’ variates, better performance of biomass production was obtained when using medium A, N=3 and 4, and T=1 and 3 min (bars A,3,3 and A,1,4) producing 26.2±1 g. In the case of ‘Rainier’, the greater biomass was produced when using medium A under the conditions of N=3 and 4 and T=1 and 3 min (bars A,3,3 and A,1,4), producing its best performance of 18.1 g of biomass.

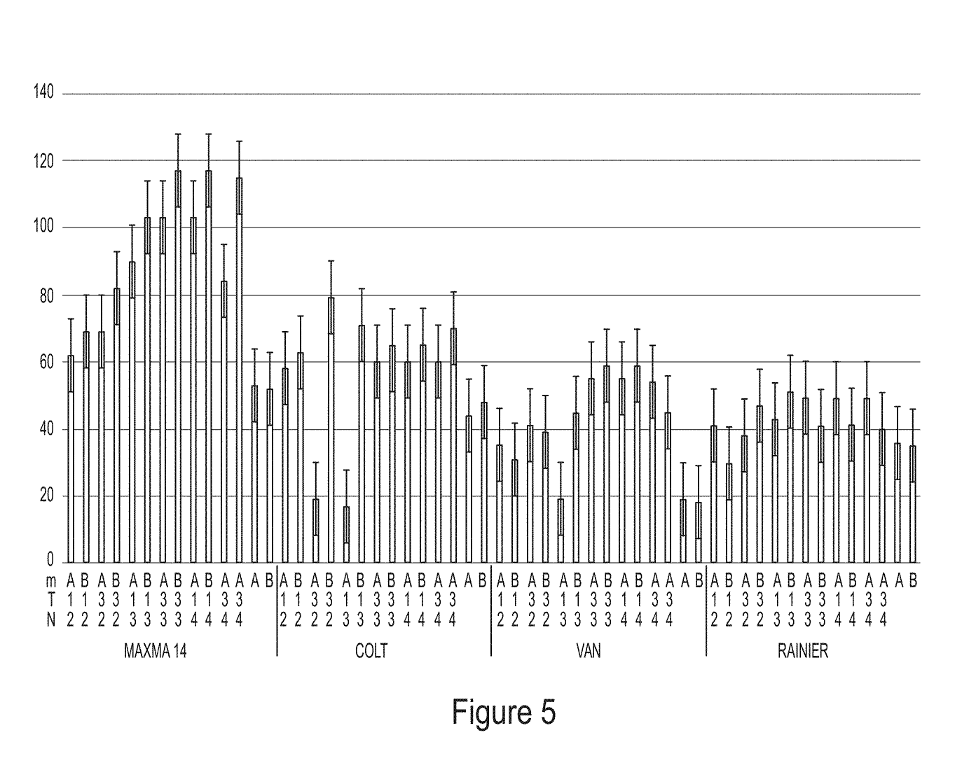

Regarding the number of seedlings produced (FIG. 5), we observed that variety MAXMA-14 showed the greatest number of seedlings under all the conditions whenever medium B was used. Additionally, we observed a good performance when the later was micro-propagated in medium A under the conditions of N=4 and T=3 min, reaching a maximum production of 117±10 y 115±10 seedling, respectively. The Colt explants had their greater number of seedlings produced with the use of medium B under conditions of N=2 for T=3 min (79±10 explants) and N=3 for T=1 min (71±10).

Micropropagation Performance Comparison Between Temporary Immersion Systems and Micropropagation in a Solid Medium.

At the same time, we compared the micropropagation performance by using the temporary immersion bioreactor system and by carrying out micropropagation of the same explants in a solid culture medium.

The solid medium used contains a DKW medium (Driver and Kuniyuki, 1984) supplemented with 0.01 mg/L IBA, 500 mg/L ascorbic acid, 100 mg/L of inositol and 7 g/L of Phytagel.

Again, the micropropagation performance was determined based on final biomass, number of seedlings produced, and sucrose consumption.

Biomass Production and Number of Seedlings

It was observed that the biomass production for genotypes MAXMA-14 and Colt rootstock is greater using the immersion system in comparison to the observations made when the micropropagation is performed in the semisolid medium. (FIG. 4).

In the case of the ‘Van’ variety, most of the cultures in the temporary immersion system showed, except for the protocol with the medium A, N=3 and T=1 min, a higher production performance in comparison to the culture in solid (FIG. 5).

With respect to the culture of the ‘Rainer” genotype, it did not show significant differences between micropropagation in the immersion system and culture in a solid medium.

Sucrose Consumption

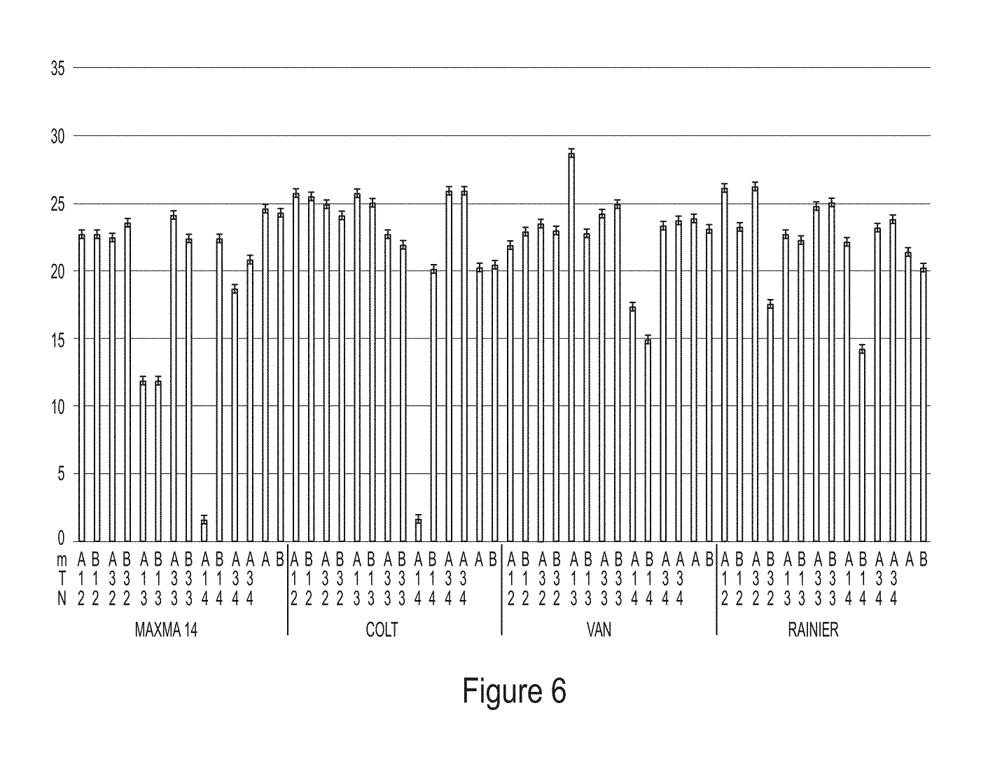

The sucrose consumption in the culture of the MAXMA-14 variety, in general, showed differences between culture in the immersion system and that in a solid medium (FIG. 6). Similarly, the Colt explants consume different amounts of sucrose in the immersion systems evaluated in comparison to the semisolid medium.

The behavior of the varieties also showed the same tendency observed in the rootstock, that is, different consumptions in comparison with the micropropagation in a semisolid medium.

Finally, the ‘Van’ explants showed the same consumption of sucrose in the semisolid medium that in the 4 conditions evaluated in the immersion system.

1. An in vitro culture method to increase the biomass and the number of seedlings of different varieties and rootstocks of Prunus spp. CHARACTERIZED by including the following stages:
a) Collecting shoots from the donor plant,
b) Washing the shoots with water for 10 minutes and disinfecting them with a 50% commercial bleach solution at for 20-40 minutes,
c) Submerging the shoots in a pesticide for 20-40 minutes,
d) Submerging the shoots in an indolebutyric acid solution (IBA) for 20-40 minutes,
e) Collecting the buds and introducing them into a temporary immersion system made up of media A and B, wherein immersions are performed 2 and 8 times per day with a duration of 1 to 6 minutes for the duration of the 10-20 days culture period.
f) The in vitro seedlings are obtained after the require culture period.

2. In vitro culture method to increase the biomass and the number of seedlings of different varieties and rootstocks of Prunus spp. Pursuant to claim No. 1 CHARACTERIZED because on stage e), the temporary immersion system contains a medium B, which consists of a modified DKW base medium with a concentration of 0.001 to 0.1 mg/L of indole butyric acid (IBA), 0.1 to 3 mg/L of 6-benzylaminopurine (BAP), 25 gr/L of sucrose, 0.5 gr/L of ascorbic acid and 0.1 gr/L of inositol.

3. In vitro culture method to increase the biomass and the number of different varieties and rootstocks of Prunus spp Pursuant to claim No. 1 CHARACTERIZED because on stage e), the temporary immersion system contains a medium A, which consists of a modified DKW base medium with 0.001 to 0.01 mg/L of indole butyric acid (IBA), 0.1 mg/L of 6-benzylaminopurine (BAP), 25 gr/L of sucrose, 0.5 gr/L of ascorbic acid and 0.1 gr/L of inositol.

4. In vitro culture method to increase the biomass and the number of seedlings of different varieties and rootstocks of Prunus spp. Pursuant to claim No. 1 CHARACTERIZED because on stage e), the temporary immersion system contains a medium A, which consists of a modified DKW base medium with 0.001 to 0.01 mg/L of indole butyric acid (IBA), 0.6 mg/L of 6-benzylaminopurine (BAP), 25 gr/L of sucrose, 0.5 gr/L of ascorbic acid and 0.1 gr/L of inositol.

5. Use of the in vitro culture method to increase the biomass and the number of seedlings of different varieties and rootstocks of Prunus spp. Pursuant to claim No. 1 CHARACTERIZED by its usefulness to micropropagate in vitro any variety and rootstock of Prunus spp.

6. Use of the in vitro culture method to increase the biomass and the number of seedlings of different varieties and rootstocks of Prunus spp. Pursuant to claim No. 1 CHARACTERIZED by its usefulness to micropropagate in vitro Prunus avium.
